(19) United States
(12) Patent Application Publication
(10) Pub. No.: US 2024/0208817 A1
(43) Pub. Date: Jun. 27, 2024

(54) HEXAGONAL BORON NITRIDE POWDER AND METHOD FOR PRODUCING SAME, AND COSMETICS AND METHOD FOR MANUFACTURING SAME

(71) Applicant: Denka Company Limited, Tokyo (JP)

(72) Inventor: Ryuki MATSUI, Tokyo (JP)

(73) Assignee: Denka Company Limited, Tokyo (JP)

(21) Appl. No.: 18/567,872

(22) PCT Filed: Jun. 16, 2021
(86) PCT No.: PCT/JP2021/022907

§ 371 Date: Dec. 7, 2023

(51) Int. Cl. C01B 21/064; A61K 8/02; A61K 8/19; A61Q 19/00

(52) U.S. Cl. CPC C01B 21/0646; A61K 8/0245; A61K 8/0275; A61K 8/19; A61Q 19/00; A61K 2800/10; A61K 2800/412; C01P 2002/76; C01P 2004/50; C01P 2004/52; C01P 2006/20

(57) ABSTRACT

Provided is a hexagonal boron nitride powder including: secondary particles formed through agglomeration of primary particles of hexagonal boron nitride, in which in a cumulative distribution of volume-based particle sizes measured through a laser diffraction/light scattering method, the particle sizes at which cumulative values from small particle sizes reach 10%, 50%, and 90% of the total are respectively D10, D50, and D90, with D50 being 3 to 30 μm, and a D90/D10 ratio is 4.0 or more.

### TECHNICAL FIELD

The present disclosure relates to a hexagonal boron nitride powder and a method for producing the same, and a cosmetic preparation and a method for producing the same.

### BACKGROUND ART

Boron nitride has lubricity, high thermal conductivity, insulation properties, and the like, and is used in a wide range of applications, including solid lubricants, mold release agents, fillers for resins and rubber, raw materials for a cosmetic preparation (also called a cosmetic), and insulating sintered bodies with heat resistance.

Examples of functions of the hexagonal boron nitride powder mixed into a cosmetic preparation include improving slipperiness, spreadability, and concealability of the cosmetic preparation, and imparting glossiness to the cosmetic preparation. In particular, the hexagonal boron nitride powder has superior slipperiness compared to a talc powder and a mica powder, which have similar functions, and are therefore widely used in the cosmetic preparation that requires excellent slipperiness. Patent Literature 1 proposes that the average particle size and the maximum particle size be within a predetermined numerical range to improve slipperiness of a hexagonal boron nitride powder.

### CITATION LIST

### Patent Literature

[Patent Literature 1] Japanese Unexamined Patent Publication No. 2018-165241

### SUMMARY OF INVENTION

### Technical Problem

To meet the increasingly high level of customer demands for a cosmetic preparation, there has been a demand for further improvement in the properties of raw materials used in the cosmetic preparation. For example, it is thought that it is necessary for raw materials used for foundation and the like to have superior spreadability. To improve spreadability, it is thought that it is effective to make the powder bulky to some extent.

The present disclosure provides a hexagonal boron nitride powder that can produce a cosmetic preparation with excellent spreadability, and a method for producing the same. In addition, the present disclosure provides a cosmetic preparation with spreadability using the above-described hexagonal boron nitride powder, and a method for producing the same.

### Solution to Problem

A hexagonal boron nitride powder according to an aspect of the present disclosure includes: secondary particles formed through agglomeration of primary particles of hexagonal boron nitride, in which in a cumulative distribution of volume-based particle sizes measured through a laser diffraction/light scattering method, the particle sizes at which cumulative values from small particle sizes reach 10%, 50%, and 90% of the total are respectively D10, D50, and D90, with D50 being 3 to 30 μm, and a D90/D10 ratio is 4.0 or more.

Since the above-described hexagonal boron nitride powder has a D50 of 3 to 30 μm, it contains primary particles of a size suitable for spreadability. In this manner, although the primary particles have a size suitable for spreadability, the volume proportion of the secondary particles formed through agglomeration of the primary particles is able to have been increased because of a large D90/D10 ratio. The secondary particles can have larger voids therein compared to the primary particles. Accordingly, a hexagonal boron nitride powder with a large volume proportion of the secondary particles becomes bulky and has a fluffy appearance. The agglomerated secondary particles are spread while being destroyed when such a hexagonal boron nitride powder is spread. For this reason, the hexagonal boron nitride powder has excellent spreadability. Such a hexagonal boron nitride powder is suitable for use as a raw material for a cosmetic preparation.

The D90/D50 ratio of the above-described hexagonal boron nitride powder may be 1.7 or more. Due to this, the proportion of the secondary particles can be further increased, and an effect of improving spreadability due to the secondary particles during spreading can be further increased. Accordingly, a hexagonal boron nitride powder having superior spreadability can be obtained.

The D90 of the above-described hexagonal boron nitride powder may be 50 μm or less. This makes it possible to reduce the number of coarse particles formed through excessive agglomeration of the primary particles and reduce roughness when the hexagonal boron nitride powder is used for a raw material for a cosmetic preparation.

The above-described hexagonal boron nitride powder may be used for a raw material for a cosmetic preparation. Since the above-described hexagonal boron nitride powder has excellent spreadability, it is suitable for use as a raw material for the cosmetic preparation.

A method for producing a hexagonal boron nitride powder according to one aspect of the present disclosure includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; a firing step of firing a mixed powder containing the calcined product and an aid at 1900° C. to 2100° C. in an atmosphere of an inert gas, ammonia gas, or a mixed gas thereof to obtain a fired product containing hexagonal boron nitride having higher crystallinity than the hexagonal boron nitride of the calcined product; a purification step of washing and drying the fired product to obtain a dry powder; an annealing step of annealing the dry powder at 1900° C. to 2100° C. in an atmosphere of an inert gas, ammonia gas, or a mixed gas thereof to obtain a heat-treated product containing secondary particles formed through agglomeration of primary particles of hexagonal boron nitride; and a crushing step of crushing the heat-treated product to obtain a hexagonal boron nitride powder containing the secondary particles.

The above-described production method includes the calcination step of performing firing at a temperature lower than that in the firing step and the firing step of performing firing using an aid, thereby making it possible to form primary particles of hexagonal boron nitride with a small particle size and high crystallinity. Then, the amount of aid or the like remaining in the fired product is reduced through the purification step, and grain growth can be suppressed in the subsequent annealing step. By performing the crushing step after the annealing step, coarse particles can be crushed into secondary particles having an appropriate size while maintaining the secondary particles in which the primary particles are agglomerated.

In the thus obtained hexagonal boron nitride powder, the volume proportion of the secondary particles in which the primary particles are agglomerated is able to have been increased. The secondary particles have larger voids therein compared to the primary particles. Accordingly, a hexagonal boron nitride powder with a large volume proportion of the secondary particles becomes bulky and has a fluffy appearance. The agglomerated secondary particles are spread while being destroyed when such a hexagonal boron nitride powder is spread. For this reason, the hexagonal boron nitride powder has excellent spreadability. Such a hexagonal boron nitride powder is suitable for use as a raw material for a cosmetic preparation.

In the hexagonal boron nitride powder obtained in the crushing step of the above-described production method, when particle sizes when cumulative values from small particle sizes reach 10%, 50%, and 90% of the total are respectively D10, D50, and D90 in a cumulative distribution of volume-based particle sizes measured through a laser diffraction/light scattering method, D50 may be 3 to 30 μm and a D90/D10 ratio may be 4.0 or more.

A cosmetic preparation according to an aspect of the present disclosure includes the above-described hexagonal boron nitride powder. The above-described hexagonal boron nitride powder has excellent spreadability when spread. For this reason, the cosmetic preparation containing such hexagonal boron nitride powder have excellent spreadability.

A method for producing a cosmetic preparation according to an aspect of the present disclosure includes producing the cosmetic preparation using the hexagonal boron nitride powder obtained through any of the above-described production methods as a raw material. The hexagonal boron nitride powder obtained in the above-described production method has excellent spreadability when spread. For this reason, the cosmetic preparation produced using such a hexagonal boron nitride powder as a raw material have excellent spreadability.

### Advantageous Effects of Invention

According to the present disclosure, it is possible to provide a hexagonal boron nitride powder that can produce a cosmetic preparation with excellent spreadability, and a method for producing the same. In addition, according to the present disclosure, it is possible to provide a cosmetic preparation having excellent spreadability using the above-described hexagonal boron nitride powder, and a method for producing the same.

### BRIEF DESCRIPTION OF DRAWINGS

FIG. 1 is a graph showing a cumulative distribution of volume-based particle sizes measured through a laser diffraction/light scattering method.

### DESCRIPTION OF EMBODIMENTS

Hereinafter, embodiments of the present disclosure will be described. However, the following embodiments are merely examples for describing the present disclosure and are not intended to limit the present disclosure to the following contents.

A hexagonal boron nitride powder according to one embodiment includes: secondary particles formed through agglomeration of primary particles of hexagonal boron nitride, in which in a cumulative distribution of volume-based particle sizes measured through a laser diffraction/light scattering method, the particle sizes at which cumulative values from small particle sizes reach 10%, 50%, and 90% of the total are respectively D10, D50, and D90, with D50 being 3 to 30 μm, and a D90/D10 ratio is 4.0 or more.

D10, D50, and D90 in the present disclosure are measured using a commercially available laser diffraction particle size distribution measurement device. The magnitude relation of D10, D50, and D90 is D10<D50<D90. D50 may be 5 μm or more or 7 μm or more from the viewpoint of further improving slipperiness when the hexagonal boron nitride powder is used for a raw material for a cosmetic preparation. D50 may be 25 μm or less or 20 μm or less from the viewpoint of reducing glare in appearance when the hexagonal boron nitride powder is used for a raw material for a cosmetic preparation.

D90 may be 50 μm or less, 45 μm or less, or 40 μm or less. This makes it possible to reduce the number of coarse particles formed through excessive agglomeration of the primary particles and reduce roughness when the hexagonal boron nitride powder is used for a raw material for a cosmetic preparation. D90 may be 18 μm or more or 19 μm or more from the viewpoint of further improving spreadability. An example of a range of D90 may be 18 to 50 μm.

D10 may be 2 μm or more or 3 μm or more. Accordingly, the spreadability can be further improved. D10 may be 10 μm or less or 8 μm or less. This makes it possible to reduce glare in appearance when the hexagonal boron nitride powder is used for a raw material for a cosmetic preparation. An example of a range of D10 may be 2 to 10 μm. D10, D50, and D90 can be adjusted by, for example, the particle size distribution of a raw material powder, the calcination temperature and time, the firing temperature and time, and the annealing temperature and time.

The D90/D10 ratio may be 4.5 or more, 5.0 or more, or 6.0 or more. By increasing the D90/D10 ratio in this manner, the ratio and size of secondary particles to primary particles can be sufficiently increased. Thus, the number of voids contained in the secondary particles increases, resulting in a fluffier appearance, and the spreadability is further improved when the hexagonal boron nitride powder is spread. The upper limit of the D90/D10 ratio may be 10 or 8.0 from the viewpoint of reducing production costs. The D90/D10 ratio can be adjusted by, for example, changing the firing temperature and time in the firing step and the annealing temperature and time in the annealing step. An example of a range of the D90/D10 ratio may be 4.0 to 10.

The D90/D50 ratio may be 1.7 or more, 1.8 or more, or 2.0 or more. Due to this, the proportion of the secondary particles can be further increased, and an effect of improving spreadability due to the secondary particles during spreading can be further increased. Accordingly, a hexagonal boron nitride powder having superior spreadability can be obtained. The upper limit of the D90/D50 ratio may be 6.0 or 4.0 from the viewpoint of reducing production costs. The D90/D50 ratio can be adjusted by, for example, changing the annealing temperature and time in the annealing step. An example of a range of the D90/D50 ratio may be 1.7 to 6.0.

The D50/D10 ratio may be 4.0 or less, 3.0 or less, or 2.8 or less. This makes it possible to reduce the variation in the particle size of the primary particles and to sufficiently increase concealment. The lower limit of the D50/D10 ratio may be 1.5 or more or 2.0 or more from the viewpoint of reducing production costs. The D50/D10 ratio can be adjusted by, for example, changing the firing time in the firing step. An example of a range of the D50/D10 ratio may be 1.5 to 4.0.

The bulk density of a hexagonal boron nitride powder may be 0.45 g/cm³ or less, 0.41 cm³ or less, and 0.35 cm³ or less. By having such a low bulk density, a hexagonal boron nitride powder having a fluffier appearance can be obtained. The bulk density can be measured in accordance with “Test methods for bulk density of fine ceramic powder” in JIS R1628-1997.

In the hexagonal boron nitride according to the present embodiment, although the primary particles have a size suitable for spreadability, the volume proportion of the secondary particles formed through agglomeration of the primary particles is able to have been increased because of a large D90/D10 ratio. The secondary particles can have larger voids therein compared to the primary particles. Accordingly, a hexagonal boron nitride powder with a large number of the secondary particles becomes bulky and has a fluffy appearance. The agglomerated secondary particles are spread while being destroyed when such a hexagonal boron nitride powder is spread. For this reason, the hexagonal boron nitride powder has excellent spreadability. Such a hexagonal boron nitride powder is suitable for use as a raw material for a cosmetic preparation. That is, the present disclosure can also provide a method of using hexagonal boron nitride as a raw material for a cosmetic preparation.

A cosmetic preparation according to one embodiment contain the hexagonal boron nitride powder described above. Accordingly, the cosmetic preparation containing such a hexagonal boron nitride powder have excellent spreadability. Examples of the cosmetic preparation include foundation (powder foundation, liquid foundation, and cream foundation), face powder, point makeup, eye shadow, eyeliner, nail polish, lipstick, cheek rouge, and mascara. Among these, the hexagonal boron nitride powder is particularly well suited for foundation and eye shadow. The content of a hexagonal boron nitride powder in the cosmetic preparation is, for example, 0.1 to 70 mass %. The cosmetic preparation can be produced through a well-known method. A method for producing a cosmetic preparation includes, for example, a step of formulating and mixing a hexagonal boron nitride powder with other raw materials.

A method for producing a hexagonal boron nitride powder according to one embodiment includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; a firing step of firing a mixed powder containing the hexagonal boron nitride and an aid at 1900° C. to 2100° C. in an atmosphere of an inert gas, ammonia gas, or a mixed gas thereof to obtain a fired product containing hexagonal boron nitride having higher crystallinity than the hexagonal boron nitride of the mixed powder; a purification step of washing and drying the fired product to obtain a dry powder; an annealing step of annealing the dry powder at 1900° C. to 2100° C. in an atmosphere of an inert gas, ammonia gas, or a mixed gas thereof to obtain a heat-treated product containing secondary particles formed through agglomeration of primary particles of hexagonal boron nitride; and a crushing step of crushing the heat-treated product to obtain a hexagonal boron nitride powder containing the secondary particles.

Examples of boron-containing compounds include boric acid, boron oxide, and borax. Examples of nitrogen-containing compounds include cyandiamide, melamine, and urea. The molar ratio between boron atoms and nitrogen atoms in a raw material powder containing boron-containing compound powder and a nitrogen-containing compound powder may be boron atoms:nitrogen atoms=2:8 to 8:2, or 3:7 to 7:3. The raw material powder may contain components other than the above-described compounds. The raw material powder may contain carbonates such as lithium carbonate and sodium carbonate as a calcination aid. In addition, the raw material powder may contain a reducing substance such as carbon.

The raw material powder containing the above-described components is calcined with, for example, an electric furnace in an inert atmosphere such as nitrogen gas, helium gas, or argon gas, an ammonia atmosphere, or a mixed gas atmosphere in which these are mixed. The calcination temperature may be 600° C. to 1300° C., 800° C. to 1200° C., or 900° C. to 1100° C. The calcination time may be, for example, 0.5 to 5 hours or 1 to 4 hours.

The calcined product obtained through calcining contains at least one selected from the group consisting of low crystalline hexagonal boron nitride and amorphous hexagonal boron nitride. In the calcination step, the reaction of boron nitride is allowed to progress at a lower temperature than in the firing step to be described below. For this reason, grain growth can be suppressed, and the particle size of the primary particles of a boron nitride powder finally obtained can be reduced.

Next, the obtained calcined product is formulated and mixed with an aid to obtain a mixed powder. Examples of aids include borates such as sodium borate and carbonates such as sodium carbonate, calcium carbonate, and lithium carbonate. The formulation amount of the aid may be 2 to 20 parts by mass or 2 to 8 parts by mass based on 100 parts by mass of the calcined product containing hexagonal boron nitride. Such a mixed powder is fired in, for example, an electric furnace in an inert atmosphere such as nitrogen gas, helium gas, or argon gas, an ammonia atmosphere, or a mixed gas atmosphere in which these are mixed.

In the firing step, production and crystallization of boron nitride progress in the presence of the aid. As a result, the crystallinity of the boron nitride contained in the calcined product can be enhanced. The firing temperature is 1900° C. to 2100° C. The firing temperature may be 1950° C. to 2050° C. The firing time may be, for example, 0.5 to 5 hours or 1 to 4 hours.

If the firing temperature becomes too low, there is a trend for the secondary particles of the hexagonal boron nitride to be less likely to be produced sufficiently. If the volume proportion of the secondary particles decreases, there is a trend for slipperiness to decrease in a case where the hexagonal boron nitride is used for a raw material for a cosmetic preparation. The same trend is observed even when the firing time is too short. On the other hand, if the firing temperature is too high, the crystal growth and agglomeration of hexagonal boron nitride proceeds too much, which tends to cause strong glare when the hexagonal boron nitride is used for a raw material for a cosmetic preparation.

The fired product obtained in the firing step may contain impurities other than the hexagonal boron nitride. Examples of impurities include a residual aid and a water-soluble boron compound. In the purification step, such impurities are reduced through washing. After washing, solid-liquid separation and drying are performed to obtain a dry powder. Examples of washing liquids used for washing include water, an aqueous solution containing an acidic substance, an organic solvent, and a mixed liquid of an organic solvent and water. From the viewpoint of avoiding secondary contamination of impurities, water having an electric conductivity of 1 mS/m or less may be used. Examples of acidic substances include inorganic acids such as hydrochloric acid and nitric acid. Examples of organic solvents include water-soluble organic solvents such as methanol, ethanol, propanol, isopropyl alcohol, and acetone. The washing method is not particularly limited. For example, the fired product may be immersed in a washing liquid and stirred and washed, or the fired product may be washed by spraying a washing liquid.

After the completion of washing, the washing liquid may be subjected to solid-liquid separation through decantation or using a suction filter, a pressure filter, a rotary filter, a sedimentation separator, or a combination of these devices. A dry powder may be obtained by drying the separated solid content in a usual dryer. Examples of dryers include a shelf-type dryer, a fluidized bed dryer, a spray dryer, a rotary-type dryer, a belt-type dryer, and a combination thereof. After drying, for example, classification with a sieve may be performed to remove coarse particles.

In the annealing step, the dry powder is heated at 1900° C. to 2100° C. with, for example, an electric furnace in an inert atmosphere such as nitrogen gas, helium gas, or argon gas, an ammonia atmosphere, or a mixed gas atmosphere in which these are mixed. This annealing temperature may be 1950° C. or higher from the viewpoint of sufficiently agglomerating the primary particles. In addition, the annealing temperature may be 2050° C. or lower from the viewpoint of suppressing grain growth of the primary particles. In the annealing step, since heating is performed at a temperature equivalent to that in the firing step, a heat-treated product containing secondary particles in which primary particles are agglomerated can be obtained. The annealing time may be, for example, 0.5 to 5 hours or 1 to 4 hours from the viewpoints of sufficiently reducing the oxygen content and suppressing the grain growth.

In the crushing step, the heat-treated product obtained in the annealing step is crushed. The crushing step is preferably performed in a way that an impact is made on the agglomerated secondary particles to a degree in which the secondary particles are not destroyed. From such a viewpoint, a homogenizer applying ultrasound vibration to the heat-treated product dispersed in a solvent is preferably used in the crushing step. Those exemplified as washing liquids in the purification step can be used as the solvent. Through such a crushing step, it is possible to reduce the number of coarse particles while sufficiently leaving the secondary particles that impart a fluffy feel. In addition, impurities remaining in the heat-treated product can be smoothly washed.

The D90/D10 value of a hexagonal boron nitride powder can be adjusted by the crushing time using a homogenizer after the annealing step. Specifically, when the crushing time is increased, the D90/D10 value becomes smaller. In addition, when the crushing time is decreased, the D90/D10 value becomes larger. In this manner, the D90/D10 value can be adjusted to within a desired range by adjusting the crushing time using the homogenizer in the crushing step after performing the annealing step or the like.

The above-described hexagonal boron nitride powder can be obtained in this manner. The description regarding the embodiment of the hexagonal boron nitride powder can be applied to the above-described production method.

Some embodiments of the present disclosure have been described above, but the present disclosure is not limited to any of the above-described embodiments.

### EXAMPLES

The contents of the present disclosure will be described in more detail with reference to examples and a comparative example, but the present disclosure is not limited to the following examples.

### Example 1

### [Preparation of Hexagonal Boron Nitride Powder]

### <Calcination Step>

100.0 g of a boric acid powder (purity of 99.8 mass % or more, manufactured by Kanto Chemical Co., Inc.) and 90.0 g of a melamine powder (purity of 99.0 mass % or more, manufactured by Wako Pure Chemical Industries, Ltd.) were mixed with each other with an alumina mortar for 10 minutes to obtain a mixed raw material. The mixed raw material after drying was placed in a container made of hexagonal boron nitride and placed in an electric furnace. The temperature was raised from room temperature to 1000° C. at a rate of 10° C./min while circulating nitrogen gas in the electric furnace. After holding the temperature at 1000° C. for 2 hours, the heating was stopped and the mixture was allowed to cool naturally. The electric furnace was open at a point in time when the temperature became 100° C. or lower. In this manner, a calcined product containing low crystalline hexagonal boron nitride was obtained.

### <Firing Step>

3.0 g of sodium carbonate (purity of 99.5 mass % or more) as an aid was added to 100.0 g of the calcined product and mixed with each other with an alumina mortar for 10 minutes. The mixture was placed in the above-described electric furnace. The temperature was raised from room temperature to 2000° C. at a rate of 10° C./min while circulating nitrogen gas in the electric furnace. After holding the temperature at a firing temperature of 2000° C. for 4 hours, the heating was stopped and the mixture was allowed to cool naturally. The electric furnace was open at a point in time when the temperature became 100° C. or lower. The obtained fired product was collected and pulverized with alumina mortar for 3 minutes to obtain a coarse powder of hexagonal boron nitride.

### <Purification Step>

In order to remove impurities contained in a coarse powder of hexagonal boron nitride, 30 g of the coarse powder was put into 500 g (concentration of nitric acid: 5 mass %) of dilute nitric acid and stirred at room temperature for 60 minutes. After stirring, solid-liquid separation was performed through suction filtration, and washing was performed by replacing the liquid with water (electric conductivity of 1 mS/m) until the filtrate became neutral. After washing, the solid was dried with a dryer at 120° C. for 3 hours to obtain a dry powder.

### <Annealing Step>

The dry powder from which coarse grains were removed was placed in the above-described electric furnace. The temperature was raised from room temperature to 2000° C. at a rate of 10° C./min while circulating nitrogen gas in the electric furnace. After holding the temperature at 2000° C. for 4 hours, the heating was stopped and a fired product was allowed to cool naturally. The electric furnace was open at a point in time when the temperature became 100° C. or lower. The obtained fired product was collected to obtain a coarse hexagonal boron nitride powder.

### <Crushing Step>

30 g of the coarse hexagonal boron nitride powder obtained in the annealing step was placed in 300 mL of water and subjected to ultrasonic dispersion using a homogenizer (manufactured by Sonic & Materials, Inc., trade name: VC505) under the conditions at 500 W and 20 kHz for 5 minutes. Thereafter, to remove impurities contained in the coarse powder, 500 g (concentration of nitric acid: 5 mass %) of dilute nitric acid was added thereto and stirred at room temperature for 60 minutes. After stirring, solid-liquid separation was performed through suction filtration, and washing was performed by replacing the liquid with water (electric conductivity: 1 mS/m) until the filtrate became neutral. After washing, the solid was dried with a dryer at 120° C. for 3 hours to obtain a dry powder. Coarse grains were removed from the obtained dry powder using an ultrasonic vibrating sieve (manufactured by Kowa Kogyosho Co., Ltd., trade name: KFS-1000, mesh size of 250 μm). This powder was used as a hexagonal boron nitride powder of Example 1.

### [Evaluation of Hexagonal Boron Nitride Powder]

### <Measurement of Particle Size Distribution>

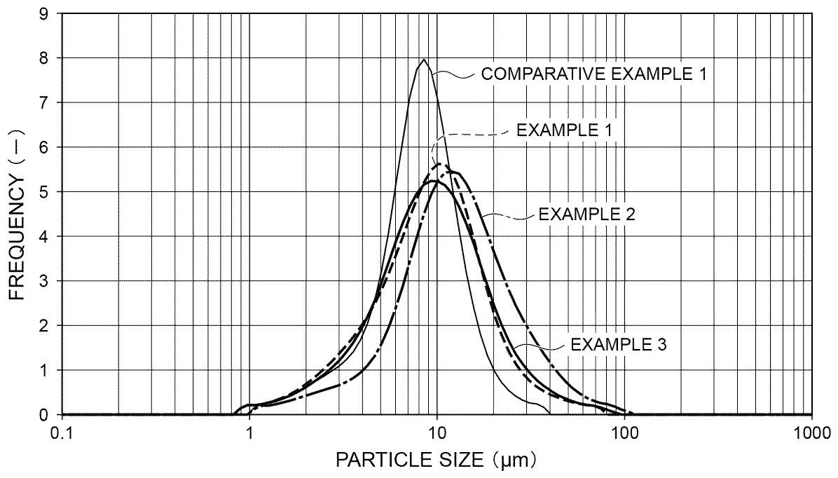

A volume-based particle size distribution of the hexagonal boron nitride powder prepared in Example 1 was measured using a laser diffraction particle size distribution measurement device (manufactured by Nikkiso Co., Ltd., device name: MT3300EX). FIG. 1 is a graph showing a cumulative distribution of volume-based particle sizes obtained through this measurement. When particle sizes when cumulative values from small particle sizes reach 10%, 50%, and 90% of the total are respectively D10, D50, and D90 in the cumulative distribution shown in FIG. 1, the values of D10, D50, and D90 are as shown in Table 2. Table 2 also shows the values of D90/D10, D90/D50, and D50/D10.

### <Evaluation for Spreadability>

0.2 g of the hexagonal boron nitride powder was placed on one end of an artificial skin (length×width=10 mm×50 mm). The hexagonal boron nitride powder was spread along the surface of the artificial skin in the vertical direction using a spatula so that the hexagonal boron nitride powder was applied to the surface. Image analysis was performed using commercially available image analysis software (WinROOF) to determine the ratio of the coating area of the hexagonal boron nitride powder to the total area of the artificial skin. The larger this area proportion, the better the spreadability. The evaluation criteria for spreadability were as shown in Table 1 depending on the area proportion. The evaluation results for spreadability are as shown in Table 2.

TABLE 1

| Area proportion | Determination |
|---|---|
| 95% or more | Very good |
| 80% or more and less than 95% | Good |
| 60% or more and less than 80% | Fair |
| 40% or more and less than 60% | Poor |
| Less than 40% | Very poor |

### Example 2

A hexagonal boron nitride powder was prepared in the same manner as in Example 1 except that the heating temperature in the annealing step was set to 2050° C. Then, various measurements and evaluations of the hexagonal boron nitride powder were performed in the same manner as in Example 1. The results are as shown in FIG. 1 and Table 2.

### Example 3

A hexagonal boron nitride powder was prepared in the same manner as in Example 1 except that the holding time in the annealing step was set to 5 hours. Then, various measurements and evaluations of the hexagonal boron nitride powder were performed in the same manner as in Example 1. The results are as shown in FIG. 1 and Table 2.

### Example 4

A hexagonal boron nitride powder was prepared in the same manner as in Example 1 except that the time for the homogenizer in the crushing step was changed to 8 minutes. Then, various measurements and evaluations of the hexagonal boron nitride powder were performed in the same manner as in Example 1. The results are as shown in Table 2.

### Comparative Example 1

A hexagonal boron nitride powder was prepared in the same manner as in Example 1 except that the heating temperature in the annealing step was set to 1700° C. Various measurements and evaluations of the hexagonal boron nitride powder were performed in the same manner as in Example 1. The results are as shown in FIG. 1 and Table 2.

TABLE 2

| | D10 | D50 | D90 | D90/ | D90/ | D50/ | |
|---|---|---|---|---|---|---|---|
| | (μm) | (μm) | (μm) | D10 | D50 | D10 | Spreadability |
| Example 1 | 3.3 | 9.0 | 19.3 | 5.9 | 2.2 | 2.7 | Very good |
| Example 2 | 4.6 | 11.7 | 28.5 | 6.2 | 2.4 | 2.5 | Good |
| Example 3 | 3.4 | 7.7 | 21.1 | 6.2 | 2.7 | 2.3 | Good |
| Example 4 | 3.0 | 9.0 | 13.5 | 4.5 | 1.5 | 3.0 | Somewhat |
| | | | | | | | good |
| Comparative | 3.1 | 7.5 | 12.0 | 3.8 | 1.6 | 2.4 | Very bad |
| Example 1 | | | | | | | |

Examples 1 to 4 all contained secondary particles in which primary particles were agglomerated. Examples 1 to 4 had a larger D90/D10 value than Comparative Example 1 and had a fluffy appearance. For this reason, Examples 1 to 4 contained more secondary particles than Comparative Example 1 and exhibited better spreadability. The hexagonal boron nitride powder of Example 1 which was the most agglomerated had the best spreadability.

### INDUSTRIAL APPLICABILITY

According to the present disclosure, a hexagonal boron nitride powder that can produce a cosmetic preparation with excellent spreadability and a method for producing the same are provided. In addition, a cosmetic preparation with excellent spreadability using the above-described hexagonal boron nitride powder and a method for producing the same are provided.

1. A hexagonal boron nitride powder comprising:
secondary particles formed through agglomeration of primary particles of hexagonal boron nitride,
wherein in a cumulative distribution of volume-based particle sizes measured through a laser diffraction/light scattering method, the particle sizes at which cumulative values from small particle sizes reach 10%, 50%, and 90% of the total are respectively D10, D50, and D90, with D50 being 3 to 30 μm, and
wherein a D90/D10 ratio is 4.0 or more.

2. The hexagonal boron nitride powder according to claim 1,
wherein a D90/D50 ratio is 1.7 or more.

3. The hexagonal boron nitride powder according to claim 1,
wherein D90 is 50 μm or less.

4. The hexagonal boron nitride powder according to claim 1, which is for a raw material for a cosmetic preparation.

5. A method for producing a hexagonal boron nitride powder, the method comprising:
a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride;
a firing step of firing a mixed powder containing the calcined product and an aid at 1900° C. to 2100° C. in an atmosphere of an inert gas, ammonia gas, or a mixed gas thereof to obtain a fired product containing hexagonal boron nitride having higher crystallinity than the hexagonal boron nitride of the calcined product;
a purification step of washing and drying the fired product to obtain a dry powder;
an annealing step of annealing the dry powder at 1900° C. to 2100° C. in an atmosphere of an inert gas, ammonia gas, or a mixed gas thereof to obtain a heat-treated product containing secondary particles formed through agglomeration of primary particles of hexagonal boron nitride; and
a crushing step of crushing the heat-treated product to obtain a hexagonal boron nitride powder containing the secondary particles.

6. The method for producing a hexagonal boron nitride powder according to claim 5,
wherein, in the hexagonal boron nitride powder obtained in the crushing step, when particle sizes when cumulative values from small particle sizes reach 10%, 50%, and 90% of the total are respectively D10, D50, and D90 in a cumulative distribution of volume-based particle sizes measured through a laser diffraction/light scattering method, D50 is 3 to 30 μm and a D90/D10 ratio is 4.0 or more.

7. A cosmetic preparation comprising:
the hexagonal boron nitride powder according to claim 1.

8. A method for producing a cosmetic preparation, the method comprising:
producing the cosmetic preparation using the hexagonal boron nitride powder obtained through the method for producing according to claim 5 as a raw material.
